(19) United States
(12) Patent Application Publication
(10) Pub. No.: US 2026/0041327 A1
(43) Pub. Date: Feb. 12, 2026

(54) BIOLOGICAL INFORMATION MEASUREMENT DEVICE

(71) Applicant: OMRON HEALTHCARE Co., Ltd., Kyoto (JP)

(72) Inventors: Kentaro MORI, Kyoto (JP); Takeshi KUBO, Kyoto (JP); Yoshihide TOKKO, Kyoto (JP); Takaaki OKANISHI, Kyoto (JP)

(21) Appl. No.: 19/363,124

(22) Filed: Oct. 20, 2025

Related U.S. Application Data
(63) Continuation of application No. PCT/JP2024/002938, filed on Jan. 30, 2024.

(30) Foreign Application Priority Data
Jun. 6, 2023 (JP) 2023-093422

(51) Int. Cl. A61B 5/021; A61B 5/00; A61B 5/022; A61B 5/28; A61B 5/318

(52) U.S. Cl. CPC A61B 5/02141; A61B 5/022; A61B 5/28; A61B 5/318; A61B 5/681; A61B 5/742

(57) ABSTRACT

A biological information measurement device includes a blood pressure measurement unit with a cuff for assessing human blood pressure, and a unit for capturing other vitals. A flexible belt wraps and secures around the arm's outer circumference, positioning the cuff internally against the skin. The cuff enables inflation for pressure readings. The device features a main body with a bottom surface that contacts the arm during wear. Embedded in this bottom is a sensor that directly touches the arm to monitor additional biological data, e.g., pulse or oxygenation. Multiple cuffs align along the belt's length for enhanced accuracy. The sensor is placed at a “change suppression position” on the bottom surface, minimizing disruptions to sensor-arm contact during inflation. It lies along the extension of a resultant force vector, calculated by combining directional forces from the arm's central axis toward the peak inflation zones of each cuff, ensuring stable measurements.

### CROSS-REFERENCE TO RELATED APPLICATIONS

This application is the U.S. national stage application filed pursuant to 35 U.S.C. 365(c) and 120 as a continuation of International Patent Application No. PCT/JP2024/002938, filed Jan. 30, 2024, which application claims priority to Japanese Patent Application No. 2023-093422, filed Jun. 6, 2023, which applications are incorporated herein by reference in their entireties.

### TECHNICAL FIELD

The present invention relates to a biological information measurement device used by being attached to a human body.

### BACKGROUND ART

It has recently become common for an individual to measure information (hereinafter, also referred to as biological information) related to a body and health of the individual such as a blood pressure value and an electrocardiographic waveform on a daily basis by himself/herself by using a measurement device and utilize the measurement result for health management. For this reason, the demand for devices where importance is placed on portability has been increasing, and portable measurement devices attached to a wrist have been proposed (for example, Patent Document 1).

Patent Document 1 discloses a portable electrocardiograph measurement device that is attached to a wrist of a human body and can perform a blood pressure measurement. In this case, two electrodes for an electrocardiographic measurement are arranged in a circumferential direction on a back surface of a main body portion that is wound around the wrist by a belt and located on a back side of a hand. In the portable electrocardiograph measurement device having such a configuration, when an electrocardiographic signal is measured simultaneously with the blood pressure measurement, the main body portion is inclined by pressurizing and inflating the cuff wound around the wrist, and thus a contact area between the electrodes and the skin varies, and accuracy of the electrocardiographic signal may deteriorate.

### CITATION LIST

### Patent Literature

Patent Document 1: CN 114680852 A

### SUMMARY OF INVENTION

### Technical Problem

In view of the related art as described above, an object of the present invention is to provide a technique in which a sensor and skin are stably in contact with each other even when a cuff is inflated, thereby enabling an accurate measurement of biological information.

### Solution to Problem

In order to solve the above problems, the present invention is a biological information measurement device including a blood pressure measurement means including a cuff for measuring a blood pressure of a human body, a biological information measurement means for measuring biological information different from the blood pressure, a belt portion to be wound around and fixed to an outer circumference of an arm portion of the human body and arrange the cuff at the outer circumference of the arm portion, the cuff being provided on an inner circumferential side of the belt portion, a main body portion including a bottom portion located on a side to be in contact with the arm portion in an attachment state, and a sensor provided on the bottom portion and for measuring the biological information by coming into contact with the arm portion, wherein a plurality of the cuffs are provided to the main body portion on both sides in an extension direction of the belt portion, the sensor is arranged at a change suppression position of the bottom portion, where a change in a contact state of the sensor with respect to the arm portion due to inflation of the cuffs can be suppressed, the change suppression position being a position on the bottom portion along an extension line of a resultant vector obtained by combining vectors of forces acting through a central portion of the arm portion toward most inflated portions of the respective cuffs, the most inflated portion being a site that is inflated most in a radial direction when each of the cuffs is inflated.

According to this, even when the cuff is inflated during a blood pressure measurement, the change in the contact state between the sensor and the arm portion is suppressed, and thus the sensor and the skin are stably in contact with each other even when the cuff is inflated, and the biological information can be accurately measured. Here, the contact state includes at least one of an angle and a position of the sensor with respect to the arm portion.

In the present invention, the sensor may include two or more sensor units, and the sensor units may be arranged side by side in a direction orthogonal to an extension direction of the belt portion.

According to this, when the cuff is inflated, a force in a rotation direction with respect to the direction orthogonal to the extension direction of the belt portion acts, but the sensor units are arranged side by side in this direction, and thus a change in the contact state is suppressed.

In the present invention, the lengths of the plurality of cuffs in a circumferential direction may be substantially equal to each other, and the change suppression position faces a central portion of a site where the plurality of cuffs overlap each other at the outer circumference of the arm portion when the plurality of cuffs may be inflated.

According to this, in a case where the lengths of the plurality of cuffs in the circumferential direction are substantially equal to each other, the inflation obtained by combining the plurality of cuffs in the radial direction becomes the largest at the central portion of the site where the plurality of cuffs overlap each other when the plurality of cuffs are inflated, and thus, a force in a rotation direction about an axis in a direction orthogonal to the circumferential direction of the arm portion becomes small with respect to the sensor arranged at a position facing the central portion at the outer circumference of the arm portion, and thus, the change in the contact state of the sensor with the arm portion is suppressed. Here, “facing” at the outer circumference of the arm portion means that the change suppression position and the central portion of the site where the plurality of cuffs overlap each other are located on the same straight line passing through the central portion of the arm portion with the central portion of the arm portion interposed therebetween. Further, the case where the lengths of the plurality of cuffs in the circumferential direction are substantially equal to each other is not limited to the case where the lengths of the plurality of cuffs in the circumferential direction are completely equal to each other, and includes the case where the lengths of the plurality of cuffs in the circumferential direction are slightly different from each other due to a manufacturing error or the like, and the case where the lengths of the plurality of cuffs in the circumferential direction are equal to each other in a substantial sense is meant.

In the present invention, the lengths of the plurality of cuffs may be substantially equal to each other, in the attachment state, a gap may be formed in a circumferential direction between circumferential end portions of the plurality of cuffs, and the change suppression position may face a central portion of the gap at the outer circumference of the arm portion when the plurality of cuffs are inflated.

According to this, in the case where the lengths of the plurality of cuffs in the circumferential direction are substantially equal to each other, when the plurality of cuffs are inflated, the force in the rotation direction about the axis in a direction orthogonal to a circumferential direction of the arm portion becomes small with respect to the sensor arranged at a position facing the central portion of the gap formed between the end portions of the plurality of cuffs in the circumferential direction at the outer circumference of the arm portion, and thus the change in the contact state of the sensor with the arm portion is suppressed. Here, “facing” at the outer circumference of the arm portion means that the change suppression position and the central portion of the gap between the end portions in the circumferential direction are located on the same straight line passing through the central portion of the arm portion with the central portion of the arm portion interposed therebetween. Further, the case where the lengths of the plurality of cuffs in the circumferential direction are substantially equal to each other is not limited to the case where the lengths of the plurality of cuffs in the circumferential direction are completely equal to each other, and includes the case where the lengths of the plurality of cuffs in the circumferential direction are slightly different from each other due to a manufacturing error or the like, and the case where the lengths of the plurality of cuffs in the circumferential direction are equal to each other in a substantial sense is meant.

The present invention is a biological information measurement device including a blood pressure measurement means including a cuff for measuring a blood pressure of a human body, a biological information measurement means for measuring biological information different from the blood pressure, a belt portion to be wound around and fixed to an outer circumference of an arm portion of the human body and arrange the cuff at the outer circumference of the arm portion, the cuff being provided on an inner circumferential side of the belt portion, a main body portion including a bottom portion located on a side to be in contact with the arm portion in an attachment state, and a sensor provided on the bottom portion and for measuring the biological information by coming into contact with the arm portion, wherein the cuff is provided to the main body portion as a single cuff in an extension direction of the belt portion, and a change suppression position of the bottom portion, where the sensor is arranged and a change in a contact state of the sensor with respect to the arm portion due to inflation of the cuff can be suppressed, faces a most inflated portion that is a site where the cuff is inflated most in a radial direction at the outer circumference of the arm portion when the cuff is inflated.

According to this, in the configuration in which a single cuff is provided to the main body portion in the extension direction of the belt portion, the force in the rotation direction about the axis in the direction orthogonal to the circumferential direction of the arm portion becomes small with respect to the sensor arranged at the position facing the most inflated portion that is the site where the cuff is inflated most in the radial direction at the outer circumference of the arm portion when the cuff is inflated, and thus the change in the contact state of the sensor with the arm portion is suppressed. Here, “facing” at the outer circumference of the arm portion means that the change suppression position and the most inflated portion of the cuff are located on the same straight line passing through the central portion of the arm portion with the central portion of the arm portion interposed therebetween.

In the present invention, the sensor may include two or more sensor units, and the sensor units may be arranged side by side in a direction orthogonal to an extension direction of the belt portion.

According to this, when the cuff is inflated, a force in a rotation direction with respect to the direction orthogonal to the extension direction of the belt portion acts, but the sensor units are arranged side by side in this direction, and thus a change in the contact state is suppressed.

The present invention is a biological information measurement device including a blood pressure measurement means including a cuff for measuring a blood pressure of a human body, a biological information measurement means for measuring biological information different from the blood pressure, a belt portion to be wound around and fixed to an outer circumference of an arm portion of the human body and arrange the cuff at the outer circumference of the arm portion, the cuff being provided on an inner circumferential side of the belt portion, a main body portion including a bottom portion located on a side to be in contact with the arm portion in an attachment state, and a sensor provided on the bottom portion and for measuring the biological information by coming into contact with the arm portion, wherein the cuff is provided to the main body portion as a single cuff in an extension direction of the belt portion, and the sensor is arranged between a first change suppression position of the bottom portion that is a position on the bottom portion, where, when the cuff is inflated, a vector of a force acting from the position toward a most inflated portion that is a site where the cuff is inflated most in a radial direction passes through a central portion of the arm portion assumed to be thinnest, and a change in a contact state of the sensor with respect to the arm portion due to inflation of the cuff can be suppressed and a second change suppression position of the bottom portion that is another position on the bottom portion, where, when the cuff is inflated, the vector of the force acting toward the most inflated portion passes through the central portion of the arm portion assumed to be thickest, and the change in the contact state of the sensor with respect to the arm portion due to inflation of the cuff can be suppressed.

According to this configuration, in the configuration in which a single cuff is provided to the main body portion in the extension direction of the belt portion, the sensor is arranged between the first change suppression position of the bottom portion that is the position of the bottom portion of the main body portion where, when the cuff is inflated, the vector of the force acting from the position toward the most inflated portion that is the site where the cuff is inflated most in the radial direction passes through the central portion of the arm portion assumed to be the thinnest, and a change in a contact state of the sensor with respect to the arm portion due to inflation of the cuff can be suppressed and the second change suppression position of the bottom portion that is another position of the bottom portion where, when the cuff is inflated, the vector of the force acting toward the most inflated portion passes through a central portion of the arm portion assumed to be thickest, and a change in a contact state of the sensor with respect to the arm portion due to inflation of the cuff can be suppressed. and thus a change in the contact state between the sensor and the arm portion is suppressed regardless of thickness of the arm portion of the person to whom the biological information measurement device is attached, and thus the sensor and the skin are stably in contact with each other even when the cuff is inflated, and the biological information can be accurately measured.

A biological information measurement device including a blood pressure measurement means including a cuff for measuring a blood pressure of a human body, a biological information measurement means for measuring biological information different from the blood pressure, a belt portion to be wound around and fixed to an outer circumference of an arm portion of the human body and arrange the cuff at the outer circumference of the arm portion, the cuff being provided on an inner circumferential side of the belt portion, a main body portion including a bottom portion located on a side to be in contact with the arm portion in an attachment state, and a sensor provided on the bottom portion and for measuring the biological information by coming into contact with the arm portion, wherein the sensor includes two or more sensor units, and the sensor units are arranged side by side in a direction orthogonal to an extension direction of the belt portion.

According to this, when the cuff is inflated, the force in the rotation direction with respect to the direction orthogonal to the extension direction of the belt portion acts, but the sensor units are arranged side by side in this direction, and thus the change in the contact state is suppressed.

In the present invention, the sensor units may at least partially overlap each other when viewed from the direction orthogonal to the extension direction of the belt portion.

According to this, when the cuff is inflated, the force in the rotation direction with respect to the direction orthogonal to the extension direction of the belt portion acts, but the sensor units are arranged so as to at least partially overlap each other when viewed from the direction orthogonal to the extension direction of the belt portion, and thus the change in the contact state is suppressed.

The present invention is a biological information measurement device including a blood pressure measurement means including a cuff for measuring a blood pressure of a human body, a biological information measurement means for measuring biological information different from the blood pressure, a belt portion to be wound around and fixed to an outer circumference of an arm portion of the human body and arrange the cuff at the outer circumference of the arm portion, the cuff being provided on an inner circumferential side of the belt portion, a main body portion including a bottom portion located on a side to be in contact with the arm portion in an attachment state, and a sensor provided on the bottom portion and for measuring the biological information by coming into contact with the arm portion, wherein a plurality of the cuffs having lengths in a circumferential direction substantially equal to each other are provided to the main body portion on both sides in an extension direction of the belt portion, and the sensor faces a central portion of a site where the plurality of cuffs overlap each other at the outer circumference of the arm portion.

According to this, in the configuration in which the plurality of cuffs having the lengths in the circumferential direction substantially equal to each other are provided to the main body portion on both sides in the extension direction of the belt portion, the inflation in the radial direction becomes the largest at the central portion of the site where the plurality of cuffs overlap each other, and thus, the force in the rotation direction about the axis in the direction orthogonal to the circumferential direction of the arm portion becomes small with respect to the sensor arranged at the position facing the site at the outer circumference of the arm portion. Thus, the change in the contact state of the sensor with the arm portion when the cuff is inflated is suppressed. Here, “facing” at the outer circumference of the arm portion means that the sensor and the central portion of the site where the plurality of cuffs overlap each other are located on the same straight line passing through the central portion of the arm portion with the central portion of the arm portion interposed therebetween. Further, the lengths in the circumferential direction are substantially equal to each other is not limited to the case where the lengths of the plurality of cuffs in the circumferential direction are completely equal to each other, and includes the case where the lengths of the plurality of cuffs in the circumferential direction are slightly different from each other due to a manufacturing error or the like, and the lengths of the plurality of cuffs in the circumferential direction are equal to each other in a substantial sense is meant.

The present invention is a biological information measurement device including a blood pressure measurement means including a cuff for measuring a blood pressure of a human body, a biological information measurement means for measuring biological information different from the blood pressure, a belt portion to be wound around and fixed to an outer circumference of an arm portion of the human body and arrange the cuff at the outer circumference of the arm portion, the cuff being provided on an inner circumferential side of the belt portion, a main body portion including a bottom portion located on a side to be in contact with the arm portion in an attachment state, and a sensor provided on the bottom portion and for measuring the biological information by coming into contact with the arm portion, wherein a plurality of the cuffs having lengths in a circumferential direction substantially equal to each other are provided to the main body portion on both sides in an extension direction of the belt portion, in the attachment state, a gap is formed in a circumferential direction between circumferential end portions of the plurality of cuffs, and the sensor faces a central portion of the gap at the outer circumference of the arm portion.

According to this, the force in the rotation direction about the axis in a direction orthogonal to a circumferential direction of the arm portion becomes small with respect to the sensor arranged at a position facing the central portion of the gap formed between the end portions of the plurality of cuffs in the circumferential direction at the outer circumference of the arm portion, and thus the change in the contact state of the sensor with the arm portion is suppressed. Here, “facing” at the outer circumference of the arm portion means that the sensor and the central portion of the gap between the end portions in the circumferential direction are located on the same straight line passing through the central portion of the arm portion with the central portion of the arm portion interposed therebetween. Further, the lengths in the circumferential direction are substantially equal to each other is not limited to the case where the lengths of the plurality of cuffs in the circumferential direction are completely equal to each other, and includes the case where the lengths of the plurality of cuffs in the circumferential direction are slightly different from each other due to a manufacturing error or the like, and the lengths of the plurality of cuffs in the circumferential direction are equal to each other in a substantial sense is meant.

The present invention is a biological information measurement device including a blood pressure measurement means including a cuff for measuring a blood pressure of a human body, a biological information measurement means for measuring biological information different from the blood pressure, a belt portion to be wound around and fixed to an outer circumference of an arm portion of the human body and arrange the cuff at the outer circumference of the arm portion, the cuff being provided on an inner circumferential side of the belt portion, a main body portion including a bottom portion located on a side to be in contact with the arm portion in an attachment state, and a sensor provided on the bottom portion and for measuring the biological information by coming into contact with the arm portion, wherein the cuff is provided to the main body portion as a single cuff in an extension direction of the belt portion, and the sensor faces a most inflated portion where the cuff is inflated most in a radial direction at the outer circumference of the arm portion when the cuff is inflated.

According to this, in the configuration in which a single cuff is provided to the main body portion in the extension direction of the belt portion, the force in the rotation direction about the axis in the direction orthogonal to the circumferential direction of the arm portion becomes small with respect to the sensor arranged at the position facing the most inflated portion where the cuff is inflated most in the radial direction at the outer circumference of the arm portion when the cuff is inflated, and thus the change in the contact state of the sensor with the arm portion is suppressed. Here, “facing” at the outer circumference of the arm portion means that the sensor and the most inflated portion of the cuff are located on the same straight line passing through the central portion of the arm portion with the central portion of the arm portion interposed therebetween.

In the present invention, the biological information measurement means measures an electrocardiographic waveform of the human body, and the sensor is an electrode for detecting the electrocardiographic waveform.

According to this, even when the cuff is inflated during the blood pressure measurement, the change in the contact state between the electrode and the arm portion is suppressed, and thus the electrode and the skin are stably in contact with each other even when the cuff is inflated, and the electrocardiographic waveform can be accurately measured.

In the present invention, the electrode may have a shape line-symmetric with respect to a direction orthogonal to an extension direction of the belt portion.

According to this, even when a force in the rotation direction with respect to the axis in the direction orthogonal to the extension direction of the belt portion acts when the cuff is inflated, a change in the contact state between the electrode and the arm portion can be suppressed.

### Advantageous Effects of Invention

According to the present invention, the sensor and the skin are stably in contact with each other even when the cuff is inflated, and the biological information can be accurately measured.

### BRIEF DESCRIPTION OF DRAWINGS

Various embodiments are disclosed, by way of example only, with reference to the accompanying schematic drawings in which corresponding reference symbols indicate corresponding parts, in which:

FIG. 1 is an external perspective view illustrating an outline of a biological information measurement device according to an example of the present invention.

FIG. 2 is a side view illustrating the outline of the biological information measurement device according to the example.

FIG. 3 is an explanatory view illustrating an arrangement relationship when the biological information measurement device according to the example is attached to a wrist.

FIG. 4 is an external view when a main body portion of the biological information measurement device according to the example is viewed from a bottom portion side.

FIG. 5 is a block diagram illustrating a functional configuration of a biological information measurement device according to the example.

FIG. 6(A) and FIG. 6(B) are views each illustrating an arrangement of a vital sensor according to Example 1.

FIG. 7(A) and FIG. 7(B) are views each illustrating details of an arrangement of the vital sensor according to Example 1.

FIG. 8(A) and FIG. 8(B) are views each illustrating details of an arrangement of the vital sensor according to Example 2.

FIG. 9(A) and FIG. 9(B) are views each illustrating an arrangement of a vital sensor according to Example 3.

FIG. 10(A) to FIG. 10(C) are views illustrating shapes of electrodes and a distances between the electrodes according to Example 4.

FIG. 11(A) to FIG. 11(E) are views each illustrating an arrangement of electrodes according to Example 5.

### DESCRIPTION OF EMBODIMENTS

Embodiments of the present invention will be specifically described below with reference to the drawings.

### Example 1

Hereinafter, an example of the embodiments of the present invention will be described. It should be noted that the dimension, material, shape, relative arrangement, and the like of the components described in the present examples are not intended to limit the scope of this invention to them alone, unless otherwise stated.

### Device Configuration

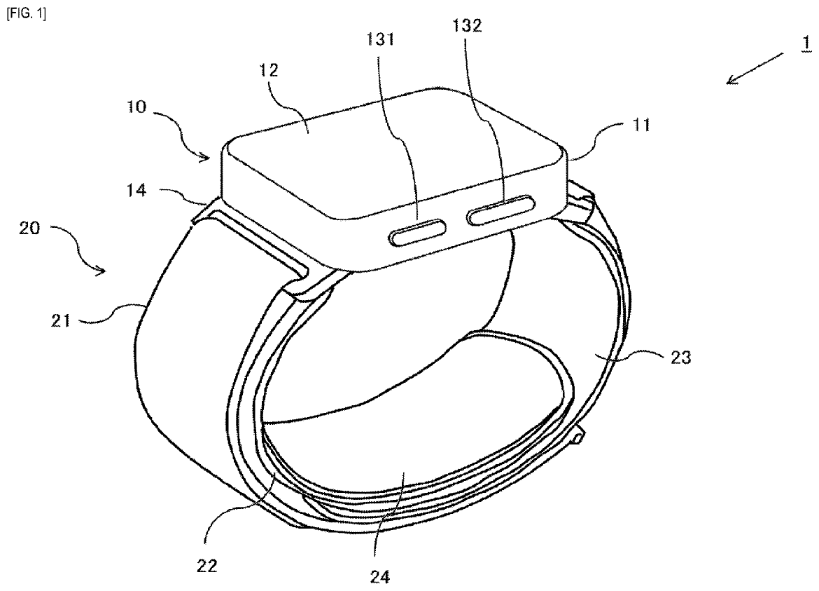

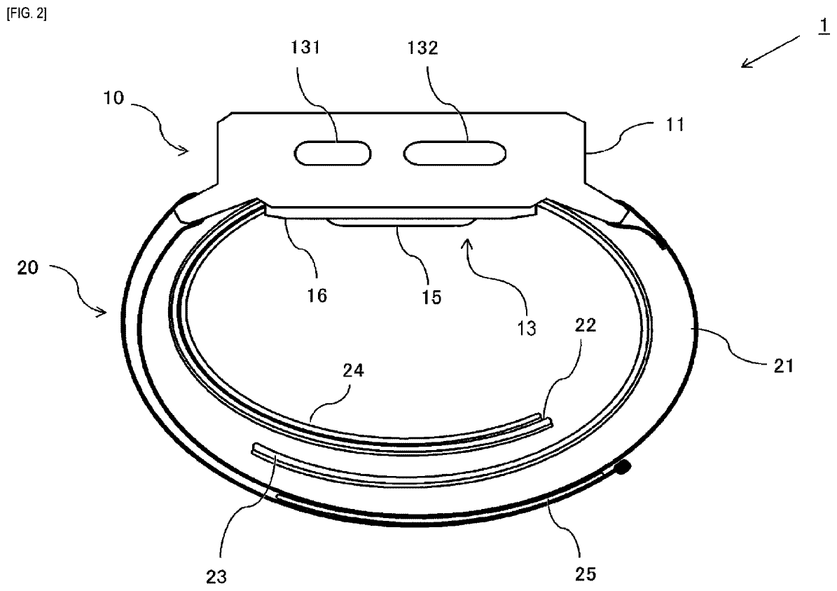

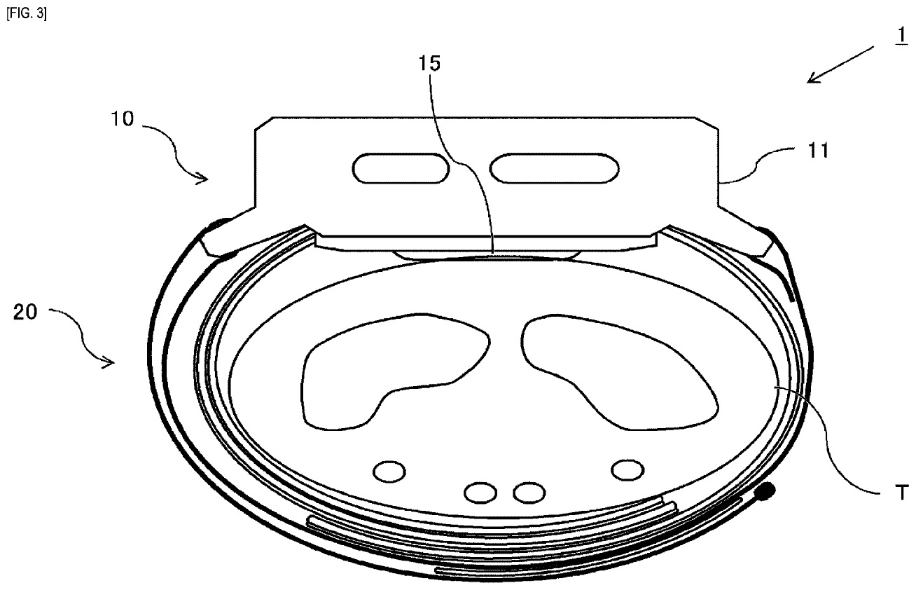

FIG. 1 is an external perspective view illustrating an outline of a configuration of a biological information measurement device 1 according to the present example. FIG. 2 is a side view illustrating the outline of the configuration of the biological information measurement device 1 according to the present example. As illustrated in FIG. 1 and FIG. 2, the biological information measurement device 1 is generally a wristwatch-type wearable device including a main body portion 10 and a belt portion 20, and can measure biological information such as a pulse wave (pulse), a blood pressure value, and an electrocardiographic waveform in a state of being attached to a wrist T of a human body. FIG. 3 illustrates an arrangement relationship between the wrist T and components of the biological information measurement device 1 according to the present example when the biological information measurement device 1 is attached to the wrist T.

As illustrated in FIG. 1 and FIG. 2, the main body portion 10 includes a main body case 11 and a cuff cover 16 described below. The main body case 11 is provided with a display 12 (for example, an organic EL display or the like), operation buttons 131 and 132, a lug 14, and the like, and is also provided with a sensor housing portion 15 in which various sensors described later are housed. Note that, in the present example, a side on which the display 12 is formed is referred to as a front surface of the main body case 11, and a side on which the sensor housing portion 15 is formed is referred to as a bottom portion 13 of the main body case 11. Note that, in the present example, the operation buttons 131 and 132 are formed of a conductor and also function as electrodes for electrocardiographic waveform measurement.

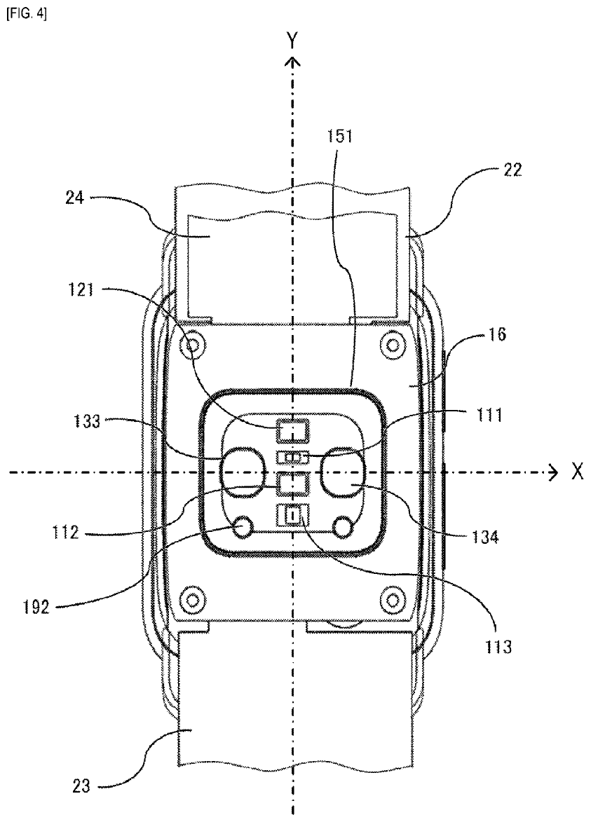

FIG. 4 is an external view of the main body portion 10 when viewed from the bottom portion 13 side. As illustrated in FIG. 4, the bottom portion 13 of the main body case 11 includes a central region covered with a light-transmissive resin cover, and a region that corresponds to the outer circumference of the central region and that is covered with the cuff cover 16. The sensor housing portion 15 is located in the central region of the main body case 11 in plan view and is covered with the light-transmissive resin cover at the bottom portion 13. Moreover, as illustrated in FIG. 2 and FIG. 3, the sensor housing portion 15 is formed so as to protrude toward the wrist T relative to the cuff cover 16 in the attachment state.

In addition, a first electrode 133 and a second electrode 134 are provided at the bottom portion 13 of the main body case 11 such that contact surfaces with the human body are exposed. One of the first electrode 133 and the second electrode 134 functions as a GND electrode during electrocardiographic waveform measurement. During the electrocardiographic waveform measurement, the biological information measurement device 1 is attached, the contact surfaces of the first electrode 133 and the second electrode 134 are brought into contact with the skin surface at the attachment portion, and the operation buttons 131 and 132 are touched with a finger on the side where the biological information measurement device 1 is not attached. Thus, the electrocardiographic waveform measurement can be performed by I induction.

In addition, a charging terminal 192 is provided at the bottom portion 13 of the main body case 11. A rechargeable battery (not illustrated in FIG. 4) can be charged by connecting a connection terminal of a power supply device and the charging terminal 192.

The belt portion 20 includes a belt 21 and a surface fastener 25 for fixing the biological information measurement device 1 to the wrist T, and also includes a first pressing cuff 22 and a second pressing cuff 23 for compressing an artery in the wrist T, and a sensing cuff 24 for detecting a pressure pulse wave. Note that connection portions between the cuffs 22, 23, and 24, and the main body case 11 are covered with the cuff cover 16. The cuff cover 16 protects the connection portions between the cuffs 22, 23, and 24, and the main body case 11, and also has a function of fixing the cuffs 22, 23, and 24 to the main body case 11. The first pressing cuff 22 is provided to the main body portion 10 on one side in the circumferential direction, and the second pressing cuff 23 is provided to the main body portion 10 on the other side in the circumferential direction. The belt 21 corresponds to the belt portion of the present invention, and the first pressing cuff 22 and the second pressing cuff 23 correspond to the plurality of cuffs of the present invention. Further, the wrist T corresponds to the arm portion of the present invention.

In addition, a processor such as a CPU, a memory such as a RAM, and the like (not illustrated) are mounted at the control board 17, and the control board 17 performs entire control of the biological information measurement device 1.

Note that, in the present example, the first LED 111 emits green irradiation light, and the second LED 113 emits red light and/or infrared light in addition to green light.

On the other hand, although not illustrated, a capacitor, an amplifier circuit, an analog-to-digital (A/D) conversion circuit, and the like are mounted at the first sensor substrate 101.

### Functional Configuration of Device

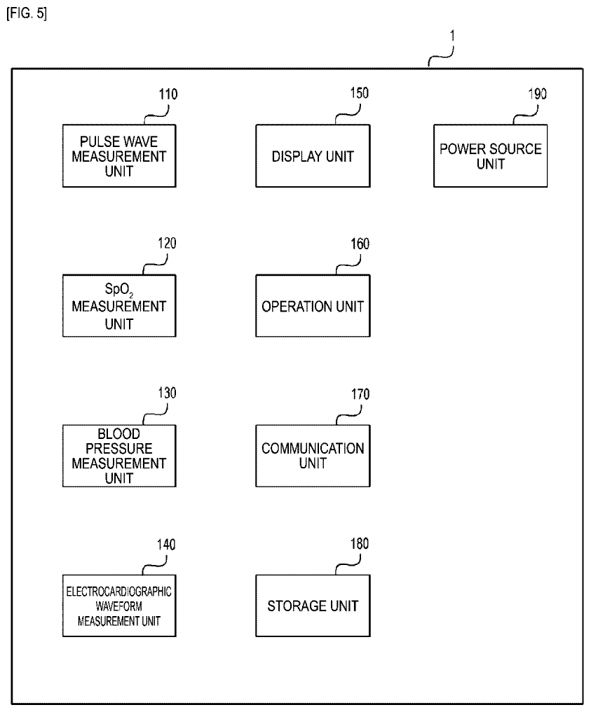

Next, a functional configuration of the biological information measurement device 1 will be described. FIG. 5 is a block diagram illustrating the functional configuration of the biological information measurement device 1. As illustrated in FIG. 5, the biological information measurement device 1 according to the present example includes functional units of a pulse wave measurement unit 110, a blood oxygen saturation (SpO2) measurement unit 120, a blood pressure measurement unit 130, an electrocardiographic waveform measurement unit 140, a display unit 150, an operation unit 160, a communication unit 170, a storage unit 180, and a power source unit 190. The processor of the control board 17 reads a program from the memory and executes the program to control each configuration of the biological information measurement device 1, thereby implementing these functional units.

The pulse wave measurement unit 110 includes the first LED 111, the second LED 113, and the first PD 112, and measures a pulse wave by a so-called photoplethysmographic method to calculate a pulse. Specifically, green light is emitted from the first LED 111 and the second LED 113, and reflection light reflected in the living body is received by the first PD 112, so that a blood flow volume that changes with a heartbeat (change in volume of a blood vessel) is detected, and a pulse wave is measured. The pulse wave corresponds to the biological information of the present invention, and the pulse wave measurement unit 110 corresponds to the biological information measurement means of the present invention. The first LED 111, the second LED 113, and the first PD 112 correspond to the sensor and the sensor unit of the present invention.

The SpO2 measurement unit 120 includes the second LED 113 and the second PD 121, and the second PD 121 receives reflection light of red light or infrared light emitted from the second LED 113, so that the SpO2 measurement unit 120 measures blood oxygen saturation from the intensity of the reflection light. The blood oxygen saturation corresponds to the biological information of the present invention, and the SpO2 measurement unit 120 corresponds to the biological information measurement means of the present invention. The second LED 113 and the second PD 121 correspond to the sensor and the sensor unit of the present invention.

The blood pressure measurement unit 130 includes a piezoelectric pump, a valve, a pressure sensor, and a flow path plate (not illustrated), and the first pressing cuff 22, the second pressing cuff 23, and the sensing cuff 24, and measures a blood pressure by a so-called oscillometric method. Since the blood pressure measurement by the oscillometric method is a well-known technology, a detailed description thereof will be omitted. The blood pressure measurement unit 130 corresponds to the blood pressure measurement means of the present invention.

The electrocardiographic waveform measurement unit 140 includes the operation buttons 131 and 132, the first electrode 133 and the second electrode 134 provided at the bottom portion 13 of the main body case 11, and an electrocardiographic waveform measuring circuit (not illustrated), and measures an electrocardiographic waveform by a so-called I-induction method. Specifically, an electrocardiographic waveform is measured based on a potential difference between the first electrode 133 and the second electrode 134 that are in contact with the wrist T of one arm in the attachment state, and a finger of the other hand in contact with the operation button 131 or 132 functioning as an electrode. The electrocardiographic waveform measurement unit 140 corresponds to the biological information measurement means of the present invention. The operation buttons 131 and 132, the first electrode 133 and the second electrode 134 correspond to the sensor and the sensor unit of the present invention.

The display unit 150 includes the display 12, and displays various types of information such as a measurement result of biological information and a menu screen. The operation unit 160 includes the operation buttons 131 and 132, and receives an input operation by the user via these buttons. The communication unit 170 includes an antenna for wireless communication (not illustrated), and communicates with another electronic device such as an information processing terminal by, for example, BLE communication. Note that a terminal for wired communication may be provided.

The storage unit 180 includes a main storage device (not illustrated) such as a random access memory (RAM) and stores various types of information such as application programs and measured biological information. In addition to the RAM, for example, a long-term storage medium such as a flash memory may be provided. The power source unit 190 includes the rechargeable battery and the charging terminal 192, and functions as a power supply source to each unit constituting the biological information measurement device 1.

### (Arrangement of Vital Sensor)

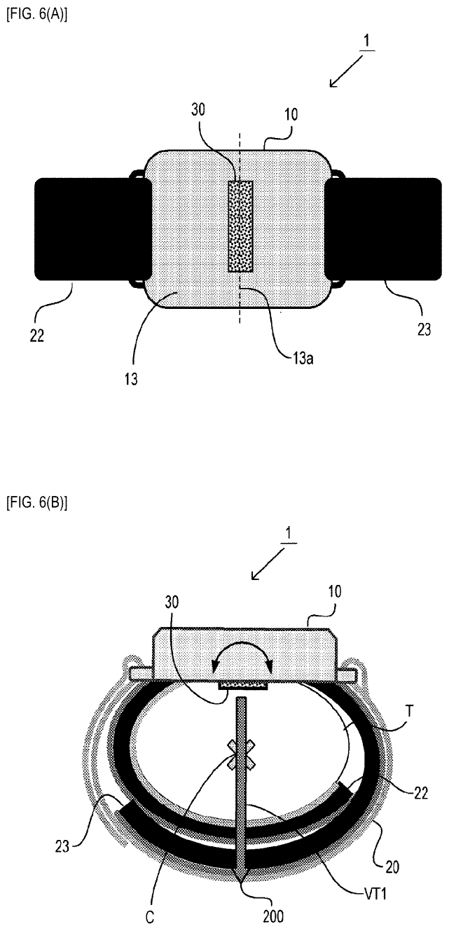

Hereinafter, the arrangement of a vital sensor 30 on the bottom portion 13 of the main body portion 10 of the biological information measurement device 1 according to the present example will be described. The vital sensor 30 is a sensor for measuring the biological information by coming into contact with the wrist T of the human body when the biological information measurement device 1 is attached. For example, the first electrode 133 and the second electrode 134 for measuring the electrocardiographic waveform described with reference to FIG. 4 correspond to the vital sensor 30, but the disclosure is not limited thereto. Although only one vital sensor 30 is illustrated in FIG. 6(A) and FIG. 6(B), this is a schematic illustration of the vital sensor 30, and two vital sensors such as the first electrode 133 and the second electrode 134 described above may be included, or the number of vital sensors is not limited in a case where the vital sensor 30 includes a plurality of the vital sensors. Here, the vital sensor 30 corresponds to the sensor of the present invention.

FIG. 6(A) is a view schematically illustrating the configuration of the biological information measurement device 1 as viewed from the bottom portion 13 side. Here, as described with reference to FIGS. 1 to 4, in the biological information measurement device 1, the vital sensor 30 having a rectangular shape and extending in a direction (X direction illustrated in FIG. 4, hereinafter also referred to as a lateral direction) orthogonal to an extension direction (Y direction illustrated in FIG. 4, hereinafter also referred to as a longitudinal direction) of the belt portion 20 of the main body portion 10 is arranged in a substantially central portion of the bottom portion 13 in the extension direction of the belt portion 20.

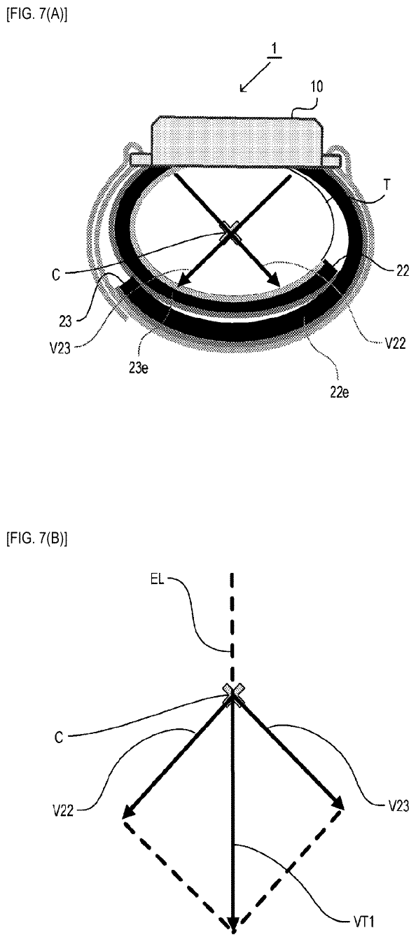

The technical significance of a position 13a (the position indicated by the broken line in FIG. 6(A)) of the bottom portion 13 in the biological information measurement device 1 will be described with reference to FIG. 7(A). In a state where the biological information measurement device 1 described here is attached to the wrist T, the first pressing cuff 22 and the second pressing cuff 23 overlap each other on the inner side of the wrist T facing the main body portion 10 with the wrist T interposed therebetween.

A most inflated portion 22e, that is a site where the first pressing cuff 22 is inflated most in the radial direction when the first pressing cuff 22 is pressurized, and similarly, a most inflated portion 23e, that is a site where the second pressing cuff 23 is inflated most in the radial direction when the second pressing cuff 23 is pressurized are respectively assumed to be the positions illustrated in FIG. 7(A). At this time, a force acting on the main body portion 10 via the belt portion 20 due to the inflation of the first pressing cuff 22 is indicated by a vector V22 passing through a central portion C of the wrist T toward the most inflated portion 22e, and a force acting on the main body portion 10 via the belt portion 20 due to the inflation of the second pressing cuff 23 is indicated by a vector V23 passing through the central portion C of the wrist T toward the most inflated portion 23e. At this time, the force acting on the main body portion 10 due to the inflation of the first pressing cuff 22 and the second pressing cuff 23 when the cuffs are pressurized can be represented by a resultant vector VT1 obtained by combining the vector V22 and the vector V23 as illustrated in FIG. 7(B). The resultant vector VT1 is also a vector passing through the central portion C of the wrist T. By providing the vital sensor 30 at a position where an extension line EL of the resultant vector VT1 representing the force acting on the main body portion 10 due to the inflation of the first pressing cuff 22 and the second pressing cuff 23 and the bottom portion 13 of the main body portion 10 intersect with each other, even when a rotational moment around an axis in the X direction due to the force acting on the vital sensor 30 due to the inflation of the first pressing cuff 22 and the second pressing cuff 23 when the cuffs are pressurized occurs, the rotational moment can be minimized. Thus, even when the first pressing cuff 22 and the second pressing cuff 23 are inflated, the rotation of the vital sensor 30 around the axis in the X direction is suppressed, and thus, even when the blood pressure measurement and the measurement of the biological information by the vital sensor 30 are performed simultaneously in parallel, a change in the contact state of the vital sensor 30 with respect to the wrist T is suppressed, and a change in the contact area between the vital sensor 30 and the wrist T is suppressed, and thus, accurate measurement can be performed. The central portion C of the wrist T is, for example, a center when a cross section of the wrist T taken in the direction of the arm is approximated by a circle or an ellipse, but the disclosure is not limited thereto. Here, the position where the extension line EL of the resultant vector VT and the bottom portion 13 of the main body portion 10 intersect with each other corresponds to the change suppression position of the present invention.

The biological information measurement device 1 illustrated in FIG. 6(A) and FIG. 6(B) illustrates an example in which the length of the first pressing cuff 22 in the circumferential direction and the length of the second pressing cuff 23 in the circumferential direction are substantially equal to each other. In such a configuration, since the vector V22 and the vector V23 described above are symmetrical with respect to a straight line connecting a central portion 200 of a site where the first pressing cuff 22 and the second pressing cuff 23 overlap each other and the central portion C of the wrist T, as illustrated in FIG. 6(B), the resultant vector VT1 overlaps the straight line connecting the central portion 200 of the site where the first pressing cuff 22 and the second pressing cuff 23 overlap each other and the central portion C of the wrist T. The position where the extension line EL of the resultant vector VT1 and the bottom portion 13 intersect with each other illustrated in FIG. 7(B) is the position 13a illustrated in FIG. 6(A). As described above, the position 13a is on the straight line connecting the central portion 200 of the site where the first pressing cuff 22 and the second pressing cuff 23 overlap each other and the central portion C of the wrist T, that is, the position 13a faces the central portion 200 of the site where the first pressing cuff 22 and the second pressing cuff 23 overlap each other at the outer circumference of the wrist T. Thus, even when the first pressing cuff 22 and the second pressing cuff 23 are inflated, the rotation of the vital sensor 30 around the axis in the X direction is suppressed, and thus, even when the blood pressure measurement and the measurement of the biological information by the vital sensor 30 are performed simultaneously in parallel, a change in the contact state of the vital sensor 30 with respect to the wrist T is suppressed, and a change in the contact area between the vital sensor 30 and the wrist T is suppressed, and thus, accurate measurement can be performed. Here, the position 13a corresponds to the change suppression position of the present invention, and the central portion 200 corresponds to the central portion of the site where the plurality of cuffs of the present invention overlap each other. Further, the case where the length of the first pressing cuff 22 in the circumferential direction and the length of the second pressing cuff 23 in the circumferential direction are substantially equal to each other is not limited to the case where the lengths in the circumferential direction are completely equal to each other, and includes the case where the lengths in the circumferential direction are slightly different from each other due to a manufacturing error or the like, and the case where the lengths in the circumferential direction are equal to each other in a substantial sense is meant.

### Example 2

Hereinafter, a biological information measurement device 1-2 according to Example 2 will be described. The biological information measurement device 1-2 has a common configuration except that the lengths are different from those of the biological information measurement device 1 according to Example 1 although the length of the first pressing cuff 22 in the circumferential direction and the length of the second pressing cuff 23 in the circumferential direction are substantially equal to each other, as described below. Configurations common to those in Example 1 are denoted by the common reference numerals, and detailed description thereof will be omitted. Here, the lengths in the circumferential direction are substantially equal to each other is not limited to the case where the lengths in the circumferential direction are completely equal to each other, and includes the case where the lengths in the circumferential direction are slightly different from each other due to a manufacturing error or the like, and the case where the lengths in the circumferential direction are equal to each other in a substantial sense is meant.

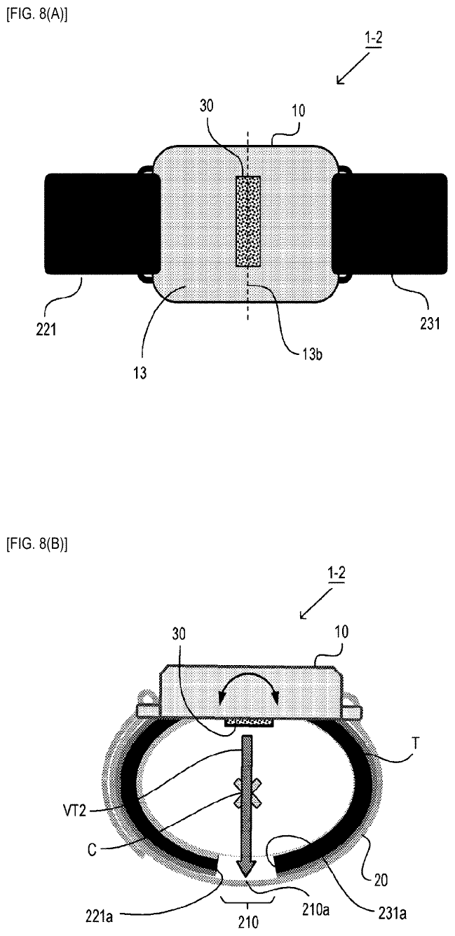

As illustrated in FIG. 8(A), the biological information measurement device 1-2 includes a first pressing cuff 221 corresponding to the first pressing cuff 22 and a second pressing cuff 231 corresponding to the second pressing cuff 23. As illustrated in FIG. 8(B), in the attachment state in which the biological information measurement device 1-2 is fixed to the wrist T by the belt portion 20, the first pressing cuff 221 and the second pressing cuff 231 wound around the outer circumference of the wrist T do not have an overlapping portion, and a gap 210 in which no cuff is present at the outer circumference of the wrist T is present between a circumferential end portion 221a of the first pressing cuff 221 and a circumferential end portion 231a of the second pressing cuff 231. Here, a position 13b where the vital sensor 30 is arranged on the bottom portion 13 is a position facing a central portion 210a of the gap 210 with the wrist T interposed therebetween. The gap 210 corresponds to the gap of the present invention. The position 13b corresponds to the change suppression position of the present invention.

During the blood pressure measurement, a resultant vector VT2 obtained by combining a vector passing through the central portion C of the wrist T toward the most inflated portion of the first pressing cuff 221 and a vector passing through the central portion C of the wrist T toward the most inflated portion of the second pressing cuff 231 overlaps a straight line connecting the central portion 210a of the gap 210 and the central portion C of the wrist T. Here, a position where an extension line of the resultant vector VT2 and the bottom portion 13 intersect with each other is the position 13b. The position 13b is on the straight line connecting the central portion 210a of the gap 210 and the central portion C of the wrist T, that is, the position 13b faces the central portion 210a of the gap 210 formed in the circumferential direction between the circumferential end portion 221a of the first pressing cuff 221 and the circumferential end portion 231a of the second pressing cuff 231 at the outer circumference of the wrist T. Thus, even when the first pressing cuff 221 and the second pressing cuff 231 are inflated, the rotation of the vital sensor 30 around the axis in the X direction is suppressed, and thus, even when the blood pressure measurement and the measurement of the biological information by the vital sensor 30 are performed simultaneously in parallel, a change in the contact state of the vital sensor 30 with respect to the wrist T is suppressed, and a change in the contact area between the vital sensor 30 and the wrist T is suppressed, and thus, accurate measurement can be performed.

### Example 3

Hereinafter, a biological information measurement device 1-3 according to Example 3 will be described. The biological information measurement device 1-3 has a configuration common to the biological information measurement device 1 according to Example 1 except that only one pressing cuff 222 is provided to the main body portion 10 as described below. Configurations common to those in Example 1 are denoted by the common reference numerals, and detailed description thereof will be omitted.

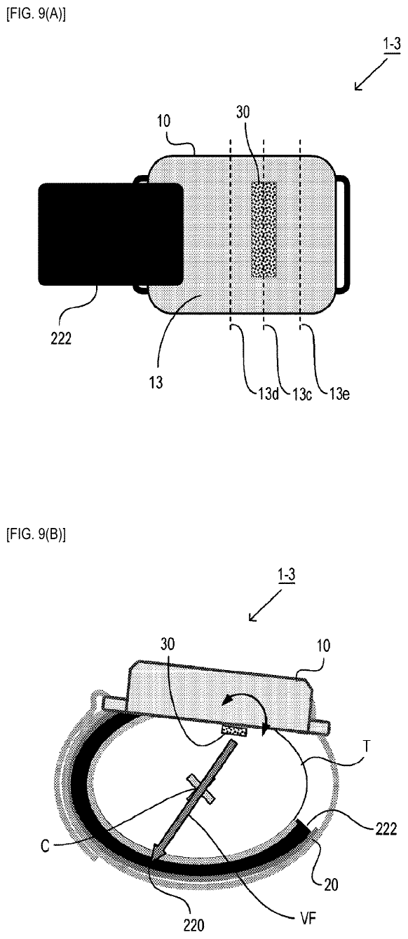

As illustrated in FIG. 9(A), the biological information measurement device 1-3 includes the pressing cuff 222 corresponding to the first pressing cuff 22, but does not include a pressing cuff corresponding to the second pressing cuff 23. As illustrated in FIG. 9(B), in a state where the biological information measurement device 1-3 is fixed to the wrist T by the belt portion 20, only a part of the outer circumference of the wrist T is pressed by the inflation of the pressing cuff 222 wound around the outer circumference of the wrist T. At this time, a site where the pressing cuff 222 is inflated most in the outer diameter direction is a most inflated portion 220. Here, a position 13c where the vital sensor 30 is arranged on the bottom portion 13 is a position facing the most inflated portion 220 with the wrist T interposed therebetween. When the pressing cuff 222 is inflated during the blood pressure measurement, the pressing cuff 222 is inflated to the outermost diameter side in the most inflated portion 220, and thus a force indicated by a vector VF in an arrow direction passing through the central portion C of the wrist T acts on the main body portion 10 by the pressing cuff 222 and the belt portion 20. A position where an extension line of the vector VF and the bottom portion 13 of the main body portion 10 intersect with each other is the position 13c. Since the vital sensor 30 is arranged at the position 13c facing the most inflated portion 220 with the wrist T interposed therebetween at the bottom portion 13, even when the rotational moment around the axis in the X direction occurs, the rotational moment is minimized. Thus, even when the pressing cuff 222 is inflated, the rotation of the vital sensor 30 around the axis in the X direction is suppressed, and thus, even when the blood pressure measurement and the measurement of the biological information by the vital sensor 30 are performed simultaneously in parallel, a change in the contact state of the vital sensor 30 with respect to the wrist T is suppressed, and a change in the contact area between the vital sensor 30 and the wrist T is suppressed, and thus, accurate measurement can be performed. Here, the position 13c corresponds to the change suppression position of the present invention.

Here, a relative positional relationship between the most inflated portion 220 of the pressing cuff 222 and the central portion C of the wrist T changes depending on the thickness of the wrist T of a person who uses the biological information measurement device 1-3. Thus, the position where the extension line of the vector VF of the force passing through the central portion C of the wrist T toward the most inflated portion 220 and the bottom portion 13 of the main body portion 10 intersect with each other also changes depending on the size of the wrist T of the person who uses the biological information measurement device 1. In FIG. 9(A), a position 13d indicates a position facing the most inflated portion 220 when the biological information measurement device 1-3 is assumed to be attached to a person having the thinnest wrist T, and a position 13e indicates a position facing the most inflated portion 220 when the biological information measurement device 1-3 is assumed to be attached to a person having the thickest wrist T. Thus, by arranging the vital sensor 30 in the region between the position 13d and the position 13e, even when the pressing cuff 222 is inflated, the rotation of the vital sensor 30 around the axis in the X direction is suppressed, and thus, even when persons having various thicknesses of the wrists T perform the blood pressure measurement and the measurement of the biological information by the vital sensor 30 simultaneously in parallel, a change in the contact state of the vital sensor 30 with respect to the wrist T is suppressed, and a change in the contact area between the vital sensor 30 and the wrist T is suppressed, and thus, accurate measurement can be performed. Here, the position 13d corresponds to a first change suppression position of the present invention, and the position 13e corresponds to a second change suppression position of the present invention.

### Example 4

Hereinafter a biological information measurement device 1-4 according to Example 4 will be described. In the biological information measurement device 1-4, the shape of the electrode as an example of the vital sensor 30 has a specific shape. Any of the configurations of the biological information measurement devices 1, 1-2, and 1-3 according to Example 1, Example 2, and Example 3 can be applied to an overall configuration except for the shape of the electrode of the biological information measurement device 1-4, and thus regarding the configuration other than the electrode, configurations common to those in the examples are denoted by the common reference numerals, and the detailed description thereof will be omitted.

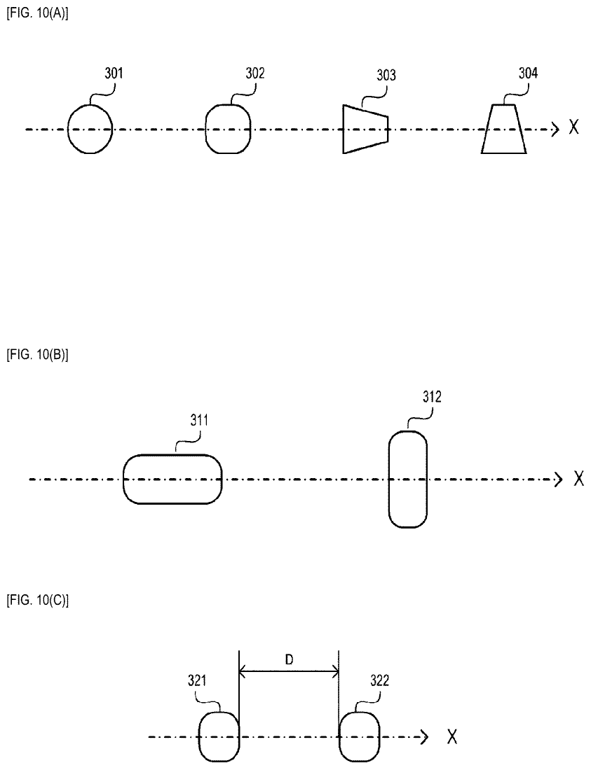

FIG. 10(A) illustrates examples of the shapes of the electrodes as the example of the vital sensor 30. An alternate long and short dash line illustrated in FIG. 10(A) is the X-axis illustrated in FIG. 4. Electrodes 301 to 303 are electrodes each having a preferable shape. The electrode 301 has a circular shape, the electrode 302 has a square shape with four rounded corners, and both have a shape line-symmetric with respect to the X-axis direction. Both the electrode 303 and the electrode 304 have a trapezoidal shape, but the electrode 303 has a shape line-symmetric with respect to the X-axis direction, whereas the electrode 304 has a shape line-symmetric with respect to a direction (Y-axis direction) orthogonal to the X-axis. Since a force in the rotation direction about the X-axis direction acts on the main body portion 10 due to the inflation of the cuff during the blood pressure measurement, when the electrodes have the shape line-symmetric in the X-axis direction as in the electrodes 301 to 303, a change in the contact area between the wrist T and the electrodes during the rotation of the main body portion 10 and a change in the pressing force of the electrodes against the wrist can be further suppressed, and thus, even when the blood pressure measurement and the measurement of the electrocardiographic waveform by using the electrodes 301 to 303 are performed simultaneously in parallel, a change in a posture of the electrodes 301 to 303 with respect to the wrist T is further suppressed, a change in the contact area and the pressing force between the electrodes 301 to 303 and the wrist T is suppressed, and accurate measurement is possible. Although one electrode is described here, the number of electrodes constituting the vital sensor 30 can be set as appropriate.

FIG. 10(B) illustrates an example of the sizes of the electrodes. The electrodes 311 and 312 each having a substantially rectangular shape illustrated in FIG. 10(B) schematically represent the sizes of the electrodes, and specifically, have shapes as those of the electrodes 301 to 303. The electrode 311 has a larger size in the X-axis direction than a size in the direction orthogonal to the X-axis. When the electrode has such a size, a change in the contact area between the wrist T and the electrode during the rotation of the main body portion 10 and a change in the pressing force of the electrode against the wrist can be suppressed, and thus, even when the blood pressure measurement and the measurement of the electrocardiographic waveform using the electrode 311 are performed simultaneously in parallel, the change in the posture of the electrode 311 with respect to the wrist T is suppressed, the change in the contact area and the pressing force between the electrode 311 and the wrist T is further suppressed, and further accurate measurement is possible. Around the X-axis, for example, the electrode 311 may have a width from 12 mm to 13 mm in the direction orthogonal to the X-axis. In contrast, as in the electrode 312, it is also conceivable to increase the size in the direction orthogonal to the X-axis as compared with the size in the X-axis direction. However, in order to suppress the change in the contact area between the wrist T and the electrode due to the rotation of the main body portion 10 around the X-axis and the change in the pressing force of the electrode against the wrist, the size of the electrode 311 is desirable.

FIG. 10(C) illustrates a distance when a plurality of the electrodes are included. Electrodes 321 and 322 each having an approximately square shape illustrated in FIG. 10(C) are examples for describing the distance between the two electrodes, and specific shapes and sizes are as described with reference to FIG. 10(A) and FIG. 10(B). As described above, it is desirable that a distance D in the X-axis direction between the electrodes 321 and 322 arranged side by side in the X-axis direction be, for example, 40 mm or less. When the distance D between the electrodes 321 and 322 is such a distance, the contact state with the wrist T during the blood pressure measurement becomes uniform, and thus, further accurate measurement of the electrocardiographic waveform can be performed simultaneously with the blood pressure measurement. On the other hand, when the distance D between the electrodes 321 and 322 is too large, the contact state with the wrist T during the blood pressure measurement becomes uneven, and thus, there is a possibility that the accuracy of the measurement of the electrocardiographic waveform is affected.

### Example 5

Hereinafter a biological information measurement device 1-5 according to Example 5 will be described. In the biological information measurement device 1-5, electrodes are arranged at specific positions on the bottom portion 13. As described in Example 1 to Example 3, there are desirable positions with respect to the bottom portion 13, but there is a degree of freedom of the positions of the electrodes at the positions, and thus various configurations are possible with respect to the number and arrangement of the electrodes. Any of the configurations of the biological information measurement devices 1, 1-2, and 1-3 according to Example 1, Example 2, and Example 3 can be applied to an overall configuration except for the arrangement of the electrode of the biological information measurement device 1-5, and thus regarding the configuration other than the electrode, configurations common to those in the examples are denoted by the common reference numerals, and the detailed description thereof will be omitted.

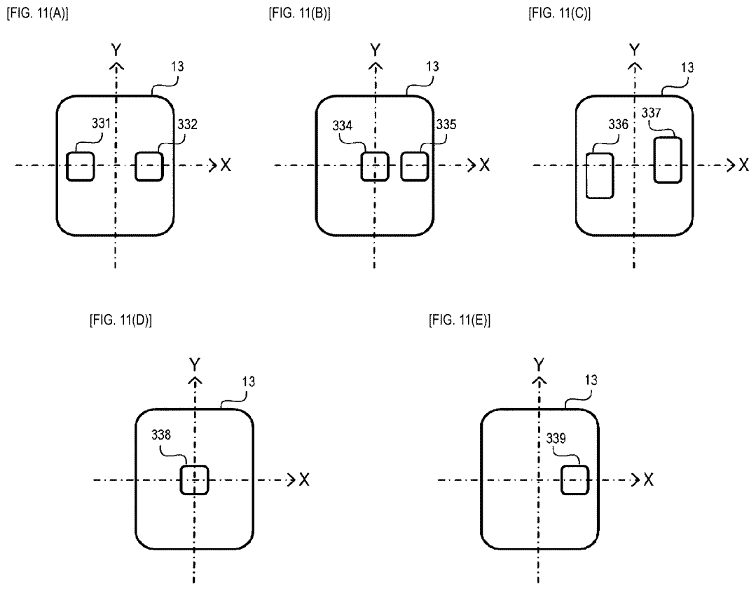

FIG. 11(A) to FIG. 11(E) each illustrate an example of the arrangement of the electrodes on the bottom portion 13 of the biological information measurement device 1-5. As illustrated in FIG. 4, the X-axis direction is an elbow-wrist direction of the person to which the biological information measurement device 1-5 is attached, and the Y-axis direction is the longitudinal direction in which the belt portion 20 extends.

In FIG. 11(A), the electrodes 331 and 332 each having a shape line-symmetric with respect to the X-axis are arranged side by side along the X-axis direction so as to be line-symmetric with respect to the Y-axis. In contrast, in the electrodes 334 and 335 illustrated in FIG. 11(B), the electrodes 331 and 332 each having a shape line-symmetric with respect to the X-axis are arranged along the X-axis direction, but the electrodes 334 and 335 are arranged at positions biased in the X direction with respect to the bottom portion 13. Not only when the electrodes are arranged evenly in the X-axis direction as in the electrodes 331 and 332, but also when the electrodes are arranged to be biased in the X-axis direction with respect to the bottom portion 13 as in the electrodes 334 and 335, the change in the contact area between the wrist T and the electrodes due to the rotation of the main body portion 10 around the X-axis and the change in the pressing force of the electrodes against the wrist can be suppressed, and accurate measurement of the electrocardiographic waveform can be performed simultaneously with the blood pressure measurement.

The electrodes 356 and 357 illustrated in FIG. 11(C) are arranged evenly in the X direction with respect to the bottom portion 13, but are respectively arranged asymmetrically with respect to the X-axis. However, when viewed in the X direction, the electrodes 356 and 357 are arranged so as to overlap each other at the central portion in the Y direction. Even with such an arrangement, the change in the contact area between the wrist T and the electrodes due to the rotation of the main body portion 10 around the X-axis and the change in the pressing force of the electrodes against the wrist can be suppressed, and accurate measurement of the electrocardiographic waveform can be performed simultaneously with the blood pressure measurement.

In FIG. 11(D), an electrode 338 is arranged at a central portion of the bottom portion 13 in the X direction and the Y direction. In contrast, an electrode 339 illustrated in FIG. 11(E) is arranged at the central portion of the bottom portion 13 in the Y direction but is arranged at a position biased from the central portion of the bottom portion 13 in the X direction. Even with such an arrangement, the change in the contact area between the wrist T and the electrode due to the rotation of the main body portion 10 around the X-axis and the change in the pressing force of the electrode against the wrist can be suppressed, and accurate measurement of the electrocardiographic waveform can be performed simultaneously with the blood pressure measurement.

### REFERENCE NUMERALS LIST

1 Biological information measurement device
10 Main body portion
13a, 13b, 13c, 13d, 13e Position
20 Belt portion
22 First pressing cuff
22e Most inflated portion
23 Second pressing cuff
23e Most inflated portion
30 Vital sensor
110 Pulse wave measurement unit
120 SpO₂ measurement unit
130 Blood pressure measurement unit
140 Electrocardiographic waveform measurement unit
133 First electrode
134 Second electrode
T Wrist

What is claimed is:

1. A biological information measurement system comprising:
a blood pressure measurement device including a cuff configured to measure a blood pressure of a human body;
a biological information measurement device configured to measure biological information different from the blood pressure;
a belt portion to be wound around and fixed to an outer circumference of an arm portion of the human body and arrange the cuff at the outer circumference of the arm portion, the cuff being provided on an inner circumferential side of the belt portion;
a main body portion including a bottom portion located on a side to be in contact with the arm portion in an attachment state; and
a sensor provided on the bottom portion and configured to measure the biological information by coming into contact with the arm portion,
wherein a plurality of the cuffs are provided to the main body portion on both sides in an extension direction of the belt portion,
the sensor is arranged at a change suppression position of the bottom portion, where a change in a contact state of the sensor with respect to the arm portion due to inflation of the cuffs can be suppressed, the change suppression position being a position on the bottom portion along an extension line of a resultant vector obtained by combining vectors of forces acting through a central portion of the arm portion toward most inflated portions of the respective cuffs, the most inflated portion being a site that is inflated most in a radial direction when each of the cuffs is inflated.

2. The biological information measurement system according to claim 1,
wherein the sensor includes two or more sensor units, and
the sensor units are arranged side by side in a direction orthogonal to an extension direction of the belt portion.

3. The biological information measurement system according to claim 1,
wherein the lengths of the plurality of cuffs in a circumferential direction are substantially equal to each other, and
the change suppression position faces a central portion of a site where the plurality of cuffs overlap each other at the outer circumference of the arm portion when the plurality of cuffs are inflated.

4. The biological information measurement system according to claim 1,
wherein the lengths of the plurality of cuffs are substantially equal to each other,
in the attachment state, a gap is formed in a circumferential direction between circumferential end portions of the plurality of cuffs, and
the change suppression position faces a central portion of the gap at the outer circumference of the arm portion when the plurality of cuffs are inflated.

5. A biological information measurement system comprising:
a blood pressure measurement device including a cuff configured to measure a blood pressure of a human body;
a biological information measurement device configured to measure biological information different from the blood pressure;
a belt portion to be wound around and fixed to an outer circumference of an arm portion of the human body and arrange the cuff at the outer circumference of the arm portion, the cuff being provided on an inner circumferential side of the belt portion;
a main body portion including a bottom portion located on a side to be in contact with the arm portion in an attachment state; and
a sensor provided on the bottom portion and configured to measure the biological information by coming into contact with the arm portion,
wherein the cuff is provided to the main body portion as a single cuff in an extension direction of the belt portion, and
a change suppression position of the bottom portion, where the sensor is arranged and a change in a contact state of the sensor with respect to the arm portion due to inflation of the cuff can be suppressed, faces a most inflated portion that is a site where the cuff is inflated most in a radial direction at the outer circumference of the arm portion when the cuff is inflated.

6. The biological information measurement system according to claim 5,
wherein the sensor includes two or more sensor units, and
the sensor units are arranged side by side in a direction orthogonal to the extension direction of the belt portion.

7. A biological information measurement system comprising:
a blood pressure measurement device including a cuff configured to measure a blood pressure of a human body;
a biological information measurement device configured to measure biological information different from the blood pressure;
a belt portion to be wound around and fixed to an outer circumference of an arm portion of the human body and arrange the cuff at the outer circumference of the arm portion, the cuff being provided on an inner circumferential side of the belt portion;
a main body portion including a bottom portion located on a side to be in contact with the arm portion in an attachment state; and
a sensor provided on the bottom portion and configured to measure the biological information by coming into contact with the arm portion,
wherein the cuff is provided to the main body portion as a single cuff in an extension direction of the belt portion, and
the sensor is arranged between
a first change suppression position of the bottom portion that is a position on the bottom portion, where, when the cuff is inflated, a vector of a force acting from the position toward a most inflated portion that is a site where the cuff is inflated most in a radial direction passes through a central portion of the arm portion assumed to be thinnest, and a change in a contact state of the sensor with respect to the arm portion due to inflation of the cuff can be suppressed, and
a second change suppression position of the bottom portion that is another position on the bottom portion, where, when the cuff is inflated, the vector of the force acting toward the most inflated portion passes through the central portion of the arm portion assumed to be thickest, and the change in the contact state of the sensor with respect to the arm portion due to inflation of the cuff can be suppressed.

8. A biological information measurement system comprising:
a blood pressure measurement device including a cuff configured to measure a blood pressure of a human body;
a biological information measurement device configured to measure biological information different from the blood pressure;
a belt portion to be wound around and fixed to an outer circumference of an arm portion of the human body and arrange the cuff at the outer circumference of the arm portion, the cuff being provided on an inner circumferential side of the belt portion;
a main body portion including a bottom portion located on a side to be in contact with the arm portion in an attachment state; and
a sensor provided on the bottom portion and configured to measure the biological information by coming into contact with the arm portion,
wherein the sensor includes two or more sensor units, and
the sensor units are arranged side by side in a direction orthogonal to an extension direction of the belt portion.

9. The biological information measurement system according to claim 8,
wherein the sensor units at least partially overlap each other when viewed from the direction orthogonal to the extension direction of the belt portion.

10. A biological information measurement system comprising:
a blood pressure measurement device including a cuff configured to measure a blood pressure of a human body;
a biological information measurement device configured to measure biological information different from the blood pressure;
a belt portion to be wound around and fixed to an outer circumference of an arm portion of the human body and arrange the cuff at the outer circumference of the arm portion, the cuff being provided on an inner circumferential side of the belt portion;
a main body portion including a bottom portion located on a side to be in contact with the arm portion in an attachment state; and
a sensor provided on the bottom portion and configured to measure the biological information by coming into contact with the arm portion,
wherein a plurality of the cuffs having lengths in a circumferential direction substantially equal to each other are provided to the main body portion on both sides in an extension direction of the belt portion, and
the sensor faces a central portion of a site where the plurality of cuffs overlap each other at the outer circumference of the arm portion.

11. A biological information measurement system comprising:
a blood pressure measurement device including a cuff configured to measure a blood pressure of a human body;
a biological information measurement device configured to measure biological information different from the blood pressure;
a belt portion to be wound around and fixed to an outer circumference of an arm portion of the human body and arrange the cuff at the outer circumference of the arm portion, the cuff being provided on an inner circumferential side of the belt portion;
a main body portion including a bottom portion located on a side to be in contact with the arm portion in an attachment state; and
a sensor provided on the bottom portion and configured to measure the biological information by coming into contact with the arm portion,
wherein a plurality of the cuffs having lengths in a circumferential direction substantially equal to each other are provided to the main body portion on both sides in an extension direction of the belt portion,
in the attachment state, a gap is formed in a circumferential direction between circumferential end portions of the plurality of cuffs, and
the sensor faces a central portion of the gap at the outer circumference of the arm portion.

12. A biological information measurement system comprising:
a blood pressure measurement device including a cuff configured to measure a blood pressure of a human body;
a biological information measurement device configured to measure biological information different from the blood pressure;
a belt portion to be wound around and fixed to an outer circumference of an arm portion of the human body and arrange the cuff at the outer circumference of the arm portion, the cuff being provided on an inner circumferential side of the belt portion;
a main body portion including a bottom portion located on a side to be in contact with the arm portion in an attachment state; and
a sensor provided on the bottom portion and configured to measure the biological information by coming into contact with the arm portion,
wherein the cuff is provided to the main body portion as a single cuff in an extension direction of the belt portion, and
the sensor faces a most inflated portion where the cuff is inflated most in a radial direction at the outer circumference of the arm portion when the cuff is inflated.

13. The biological information measurement system according to claim 1,
wherein the biological information measurement device measures an electrocardiographic waveform of the human body, and
the sensor is an electrode configured to detect the electrocardiographic waveform.

14. The biological information measurement system according to claim 13,
wherein the electrode has a shape line-symmetric with respect to a direction orthogonal to an extension direction of the belt portion.
